(19) United States
(12) Patent Application Publication
(10) Pub. No.: US 2025/0041108 A1
(43) Pub. Date: Feb. 6, 2025

(54) FACE MASK WITH UNDER-EYE GEL PACKS

(71) Applicant: Shane Gaffney, Dublin (IE)

(72) Inventor: Shane Gaffney, Dublin (IE)

(21) Appl. No.: 18/766,822

(22) Filed: Jul. 9, 2024

Related U.S. Application Data
(60) Provisional application No. 63/529,790, filed on Jul. 31, 2023.

(51) Int. Cl. A61F 7/02; A61F 7/00

(52) U.S. Cl. CPC A61F 7/02; A61F 2007/0003; A61F 2007/0004; A61F 2007/0219; A61F 2007/023; A61F 2007/0271

(57) ABSTRACT

A face mask with under-eye gel packs for cooling a facial eye region of a wearer is provided. The mask includes a face shield for covering a portion of the face of the wearer, at least one opening to reveal the eyes of the wearer to allow the wearer to see out, an opening for revealing a nose of the wearer to allow the wearer to breathe freely. The mask further includes a support band having a first portion and a second portion that goes around the wearer's head to secure the face mask on the wearer's face, and a pair of gel solution containing packs each disposed under the eye openings on the face shield. The gel solution containing packs are configured to be cooled relative to ambient temperature before use.

### CROSS-REFERENCE TO RELATED APPLICATIONS

The present application hereby claims priority to and incorporates by reference the entirety of the disclosures of the provisional application No. 63/529,790 entitled “FACE MASK WITH UNDER-EYE GEL PACKS” filed on 31 Jul. 2023.

### TECHNICAL FIELD

The present invention relates generally to face masks. Particularly, the present invention is directed to a face mask with under-eye gel packs that provide refreshing relief to the face and eyes of the wearer.

### BACKGROUND

Strain occurs on the eyes and face when the eyes get tired from intense use, such as driving a car for extended periods, reading, or working at a computer, or lack of sleep. It is estimated that each year about 10 million people seek eye exams for computer-related eyestrain. The cooling of muscles for the eyes and face to relieve tension and stress is well known in the art, but the eyes pose additional problems as chemical-based solutions can cause greater irritation and possibly even temporary or permanent blindness.

Various forms of cooling face masks and cooling eye masks are known in the art. The existing eye masks designed to soothe the eyes are generally made of plastic and are filled with a gel-like substance that can be selectively cooled and/or heated to provide relief from any number of ailments. In operation, the gel-like substance of the mask comes in direct contact with the eyelids, or the surrounding areas of the eyes when such masks are worn by users. Alternatively, the cooling face masks are found in uses that comprise eye cut-outs in the area specifically over the eyes, thus keeping the masks from coming in direct contact with the eyelids and areas around the eyes. Some of the existing cooling eye masks are offered under the following brands Medi Grade™, Prociv®, ZNÖCUETÖD™, H|M™, and Epyz™ commonly available through known marketplaces. Further, the face masks that cover the entire face of a user are known in the art. Some of the existing face masks are offered under the following brands Medi Grade™, RelaxCoo™, MR.ICE™, SKIN GYM™, Atsuwell™ and so on.

Additionally, the use of reusable eye ice packs for relieving a user from eye strain, eye swelling, eye redness, dark circles, and smooth fine lines formed along the eyes to help users relax is known in the art. For example, brands like Coldchill™, Medi Grade™ are some of the popular brands in the known marketplaces.

Not only this, inventors in the past have come up with several cooling eye masks such as US20110307041 that provide an eye pack that does not require the need for preparatory refrigeration. Likewise, JP3117357 provides a gel pack material containing a component having a cosmetic or medicinal effect on human skin that sticks to human skin. Likewise, U.S. Pat. No. 11,771,589 describes a molded face mask for treating a user's face, the molded face mask having a rigid, inflexible polycaprolactone body molded to the user's face. This mask does not cover the user's eye and leaves it open without providing any sort of cure to the eyes.

The present inventor is aware of the above-presented products and designs and many other similar eyes and face cooling masks or under-eye cooling pads, however, these existing solutions have shortcomings. None of the above-discussed face and eye cooling masks and pads are designed to be worn like any normal face mask and yet provide under eye cooling besides cooling the face.

### SUMMARY

The present invention provides a face mask with under eye gel packs. The presented face mask can be chilled in the freezer for use as a under the eye cooling pack besides cooling the face.

The proposed cooling face mask with under-eye gel packs provides a soothing effect on tired face and eyes, fatigue, dark circles, puffiness, and wrinkles.

The present invention provides a face mask that can be used at home or as a travel mask to provide a cooling effect under the user's eyes besides cooling the face.

The present invention provides a reusable face mask that provides added comfort to a user and can be used in everyday life even while taking a nap.

According to an embodiment of the present invention, there is provided a face mask for a human wearer. The face mask comprises a face shield for covering a portion of the face of the wearer, the shield is formed of a material capable of blocking airborne pathogens, dust material, and the like, wherein the face shield comprises an outwardly facing surface and an inwardly facing surface. The face mask further includes at least one revealing opening configured to reveal the eyes of the wearer to allow the wearer to see out, an opening for revealing a nose of the wearer to allow the wearer to breath freely while wearing the mask, and a support band configured to receive ears or go over the ears of the wearer. The face mask further includes a first gel solution containing pack and a second gel solution containing pack disposed under the at least one revealing openings on the face shield between the inwardly facing surface of the face shield and under the eye portions of the wearer, the gel solution containing pack configured to be cooled relative to ambient temperature.

According to the embodiment, the support band comprising a first portion and a second portion, wherein each of the first and second portions of the support band are removably attachable to each other. The first and second portions of the support band are attachable using Velcro or other similar means. The first portion and the second portion are elastic straps or bands made of stretchy fabric.

According to the embodiment, the first gel solution containing a pack and the second gel solution containing a pack are enclosed packs containing a gel solution known in the art. The gel packs are attached to the face shield and positioned to sit directly under the eye for cooling purposes.

According to the embodiment, wherein the face shield is made up of materials selected from but not limited to TPU or PVC, clear and strong plastic or silicon material.

According to the embodiment, wherein the face shield (102) may be made using a single layer or multiple layers.

These and other important features and advantages of the present invention will become apparent from the detailed description below, in light of the accompanying drawings

### BRIEF DESCRIPTION OF THE DRAWINGS

In the drawings:

FIGS. 1-3 show schematic front views of a face mask according to an exemplary embodiment of the present invention.

FIG. 4 is a side view of the face mask of FIG. 1.

FIG. 5 shows the face mask of FIG. 1 worn by a user.

### DETAILED DESCRIPTION OF THE PREFERRED EMBODIMENTS

Before describing the present invention in detail, it should be observed that the present invention utilizes a combination of components or processes, which constitutes a face mask with cooling gel packs for application under the eyes of the wearer. Accordingly, the components or processes have been represented, showing only specific details that are pertinent for an understanding of the present invention so as not to obscure the disclosure with details that will be readily apparent to those with ordinary skill in the art having the benefit of the description herein. As required, detailed embodiments of the present invention are disclosed herein; however, it is to be understood that the disclosed embodiments are merely exemplary of the invention, which can be embodied in various forms. Therefore, specific component-level details and functional details disclosed herein are not to be interpreted as limiting, but merely as a basis for the claims and as a representative basis for teaching one skilled in the art to variously employ the present invention in virtually any appropriately detailed structure. Further, the terms and phrases used herein are not intended to be limiting but rather to provide an understandable description of the invention.

References to “one embodiment”, “an embodiment”, “another embodiment”, “one example”, “an example”, “another example” and so on, indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in an embodiment” does not necessarily refer to the same embodiment. The words “comprising”, “having”, “containing”, and “including”, and other forms thereof, are intended to be equivalent in meaning and be open-ended in that an item or items following any one of these words is not meant to be an exhaustive listing of such item or items or meant to be limited to only the listed item or items.

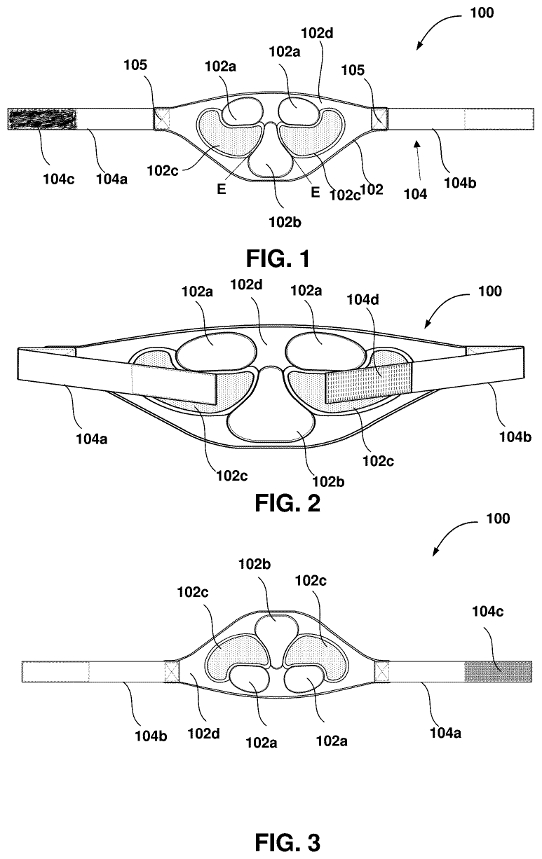

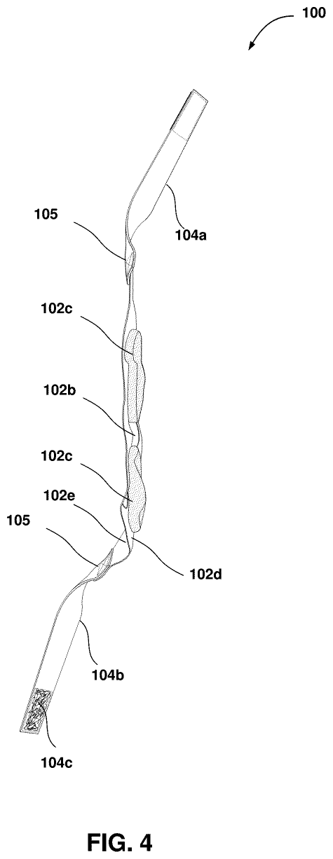

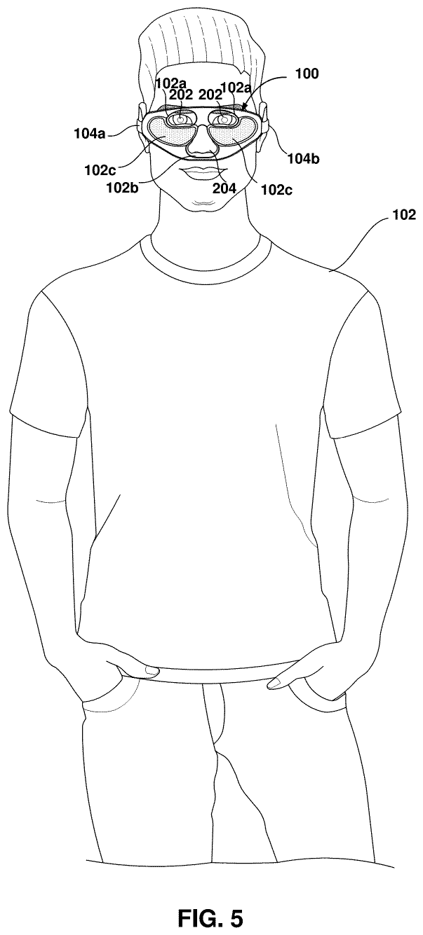

Referring to the embodiment of the face mask of the present invention along in conjunction with FIGS. 1-5, the face mask hereinafter is referred to as a mask 100. The mask 100 includes a face shield 102. The face shield 102 is configured to suit the facial anatomy of a wearer 200. In other words, the face shield 102 is sized and ergonomically shaped to cover a portion of the face of the wearer 200 and adapted to cool the facial region. The face shield 102 is formed of a material capable of blocking airborne pathogens, dust material, and the like. The face shield 102 may be made of a single layer or a single layer or multiple layers (two layers for example). The face shield 102 includes an outwardly facing surface 102d and an inwardly facing surface 102e. Although not seen in the figures, the face shield 102 is made of two layers, namely an outer layer and an inner layer, with an outer surface of the inner layer attached to the inner surface of the outer layer. Upon attachment, the two layers act as a single layer with the outwardly facing surface 102d and the inwardly facing surface 102e. The face shield 102 may be made up of but not limited to TPU or PVC, clear and strong plastic or silicon material. Usually face shield 102 is made of two layers if the face shield 102 is intended to cool the face, the multiple layers allow the face shield 102 to hold a cooling gel solution therebetween the layers. The gel solution is similar to those found in sports injury ice packs. This solution may typically consist of a glycerin and water mixture, providing an effective and lasting cooling effect. In some embodiment, the face shield area 102 may not include any cooling solution, if the face shield is not intended to cool the face and the face mask 100 is simply intended to cool the areas under the eyes.

According to the embodiment, the face shield 102 of the face mask 100 includes at least one revealing opening 102a configured to reveal the eyes 202 of the wearer 200 to allow the wearer 200 to see out of the mask 100. In the example shown, two such openings 102a are shown present, each one for a left eye and a right eye. The shapes and sizes of openings 102a are suitably chosen to just reveal the eyes 202 of the wearer 200. In some other embodiment, instead of two separate eye openings 102a, there may be one single eye opening that may be suitable for revealing both the eyes.

In an embodiment, the eye openings 102a are substantially irregular oval in shape and sized to reveal the user's eyes 202. One end of the eye opening is lesser in width compared to the other end of the eye opening 102a.

The face shield 102 further includes an opening 102b for revealing a nose 204 (referred to as “nose opening 102b”) of the wearer 200 to allow the wearer 200 to breathe freely while wearing the mask 100. The opening 102b is suitably shaped and sized to reveal the nose 204 of the wearer such as to allow comfortable breathing. In an embodiment, the nose opening 102b is made irregular in shape. It appears a pear-like in shape with an elongated end for revealing the top end of the nose 204 and a bulged out end for revealing the lower end of the nose 204 with nostrils

According to the embodiments, the face mask 100 further includes a support band 104 configured to receive ears or go over the ears of the wearer 200 to allow wearing the face mask 100 over the face of the wearer 200. The support band 104 helps in securing the face mask 100 around the wearer's head. Particularly, in a preferred embodiment, the support band 104 may include two parts, namely a first portion 104a, and a second portion 104b. The first portion 104a of the support band 104 is attached at one end/first end of the face shield 102 and the second portion 104b of the support band 104 is attached at another end/second end of the face shield 102. The first and second portions 104a, and 104b of the support band 104 are attachable to each other using Velcro (104c, 104d) or other similar means. In some embodiment, the first portion 104a and the second portion 104b may be made as one single support band 104. The first portion 104a and the second portion 104b are elastic straps or bands made of stretchy fabric. The first portion 104a and the second portion 104b are attached to the face shield 102 at two ends by stitching or sewing utilizing known techniques.

According to the embodiments, the face mask 100 further includes a first gel solution containing pack 102c and a second gel solution containing pack 102c disposed under the openings 102a located on the face shield 102. The gel solution containing packs 102c are self-contained enclosed packs disposed of or attached on the outwardly facing surface 102d of the face shield 102 under the eye openings 102a of the mask 100. The gel solution containing packs 102c include gel solution that's similar to those found in sports injury ice packs. This cooling gel solution may typically consist of a glycerin and water mixture, providing an effective and lasting cooling effect. In some other embodiment, these gel solution containing packs 102c may be disposed of on the inwardly facing surface 102e of the mask 100. The gel packs 102c are attached using adhesives or other known techniques. The gel solution containing packs 102c is configured to be cooled relative to ambient temperature. Before use, the wearer 200 can simply place the mask 100 with the packs 102c in a freezer for a certain time to cool the packs 102c attached to the face shield 102 and then the face mask 100 can be worn by the wearer 200 to receive cooling effect under the wearer's eyes. The first and second gel solution containing packs 102c comprises a thickness of about 1.4 cm protruding from the outwardly facing surface 102d of the face shield 102.

As seen, in an embodiment, each of the gel solution containing packs 102c is disposed under the eye openings 102a is irregular oval shaped complementing the shape of the eye opening 102a and the shape of the nose opening 102b. One side of each of the solution containing packs 102c, and the nose opening 102b meet together as adjacent side “E” as seen in FIG. 1.

The face mask 100 itself or any components thereof including support strap 104, gel packs 102c, and any other components may be made in different sizes and using various materials known in the art, and thus, size, the material of use should not be construed as any limitation.

Although particular embodiments of the invention have been described in detail for purposes of illustration, various modifications, and enhancements may be made without departing from the spirit and scope of the invention.

What is claimed is:

1. A face mask (100), comprising:
a face shield (102) covering a portion of a wearer's face, the face shield (102) comprising an outwardly facing surface (102d), and an inwardly facing surface (102e);
at least one first opening (102a) configured to reveal eyes (202) to allow a wearer (200) of the face mask (100) to see out;
a second opening (102b) for revealing a nose (204) to allow the wearer (200) to breathe freely while wearing the face mask (100);
a support band (104) configured for securing the face mask (100) around the wearer's head; and
a first gel solution containing pack (102c), and a second gel solution containing pack (102c) disposed on the outwardly facing surface (102d) of the face mask (100) under the at least one first opening (102a), the first and second gel solution containing packs (102c) are configured to be cooled relative to ambient temperature before use.

2. The face mask (100) of claim 1, wherein the support band (104) comprising a first portion (104a) and a second portion (104b), wherein each of the first and second portions (104a, 104b) of the support band (104) are removably attachable to each other.

3. The face mask (100) of claim 2, wherein the first and second portions (104a, 104b) of the support band (104) are attachable using a Velcro (104c, 104d).

4. The face mask (100) of claim 1, wherein the first gel solution containing pack (102c) and the second gel solution containing pack (102c) are enclosed packs that are attached to the face shield (102) using adhesive.

5. The face mask (100) of claim 1, wherein the face shield (102) is selected from a group of materials consisting of TPU, PVC, clear and strong plastic or silicon material.

6. The face mask (100) of claim 2, wherein the first portion (104a) and the second portion (104b) of the support band (104) are elastic straps or stretchy fabric.

7. The face mask (100) of claim 1, wherein the face shield (102) may be made using a single layer or multiple layers of at least TPU, PVC, clear and strong plastic or silicon material capable of blocking airborne pathogens, dust material.

8. The face mask (100) of claim 2, wherein the first portion (104a) and the second portion (104b) are fixedly attached to the face shield (102) at two ends.

9. The face mask (100) of claim 1, wherein the first and second gel solution containing packs (102c) comprises a thickness of about 1.4 cm protruding from the outwardly facing surface (102d) of the face shield (102).

10. The face mask (100) of claim 1, wherein the shape of the at least one first opening (102a) is irregular oval.

11. The face mask (100) of claim 1, wherein the shape of the first and second gel solution containing packs (102c) are irregular oval.

12. The face mask (100) of claim 1, wherein the nose opening (102b) is made irregular with a pear-like shape having an elongated end for revealing a top end of the nose (204) and a bulged-out end for revealing a lower end of the nose (204) with nostrils.

13. The face mask (100) of claim 1, wherein one side of each of the solution containing packs (102c), and the nose opening (102b) meet at a side “E”.

14. A method for cooling a facial eye region, comprising:
cooling a first and a second gel solution containing packs (102c) of a face mask (100) relative to an ambient temperature before use;
securing the face mask (100) covering a portion of a wearer's face, while revealing a wearer's eyes (202) and nose (204) such that the first and the second gel solution containing packs (102c) are disposed under at least one first opening (102a) of the face mask (100), the face mask (100) further comprising:
a face shield (102) comprising an outwardly facing surface (102d), and an inwardly facing surface (102e);
a second opening (102b) to reveal the nose (204) to allow the wearer (200) to breathe freely;
wherein, the at least one first opening (102a) is configured to reveal the eyes (202) of the wearer (200) to see out;
wherein the first and second gel solution containing pack (102c), are disposed on the outwardly facing surface (102d) of the face mask (100); and
wherein the face mask (100) is secured utilizing a support band (104) that goes around the wearer's head.

15. The method of claim 14, wherein the first and second gel solution containing packs (102c) are configured to be cooled relative to ambient temperature before use.

16. The method of claim 14, wherein the support band (104) comprising a first portion (104a) attached to an end of the face shield (102) and a second portion (104b), attached to another end of the face shield (102), wherein each of the first and second portions (104a, 104b) of the support band (104) are removably attachable to each other.

17. The method of claim 14, wherein the face shield (102) is selected from a group of materials consisting of TPU, PVC, clear and strong plastic or silicon material.

18. The method of claim 14, the first and second gel solution containing packs (102c) comprises a thickness of about 1.4 cm protruding from the outwardly facing surface (102d) of the face shield (102).

19. The method of claim 14, wherein the shape of the at least one first opening (102a) and the shape of the first and second gel solution containing packs (102c) are irregular oval.

20. The method of claim 14, wherein the nose opening (102b) is made irregular with a pear-like shape having an elongated end for revealing a top end of the nose (204) and a bulged-out end for revealing a lower end of the nose (204) with nostrils.
